(19) United States
(12) Patent Application Publication
(10) Pub. No.: US 2019/0328710 A1
(43) Pub. Date: Oct. 31, 2019

(54) SYNERGISTIC COMPOSITION FOR INHIBITING ADVANCED GLYCATION END PRODUCT CROSSLINKING

(71) Applicant: H2 Water Technologies Ltd., Vancouver (CA)

(72) Inventors: Alexander TARNAVA, Coquitlam (CA); Richard James HOLLAND, Vancouver (CA)

(21) Appl. No.: 16/395,689

(22) Filed: Apr. 26, 2019

Related U.S. Application Data
(60) Provisional application No. 62/664,136, filed on Apr. 28, 2018.

(51) Int. Cl. A61K 31/417; A61K 31/216; A61K 31/381; A61K 31/505; A61K 31/12; A61K 31/145; A61K 36/45; A61K 31/05

(52) U.S. Cl. CPC A61K 31/417; A61K 31/216; A61K 31/381; A61K 31/05; A61K 31/12; A61K 31/145; A61K 36/45; A61K 31/505

(57) ABSTRACT

The invention provides a composition or kit including two, three, or all of rosmarinic acid; alpha-lipoic acid; benfotiamine; carnosine; and optionally vitamin B6. The compositions and kits are useful for reducing AGE crosslinking or the visible signs of aging.

### BACKGROUND OF THE INVENTION

Advanced Glycation End product (AGE) crosslinking is a naturally occurring physiological phenomena. Many pathological states implicated in ago related diseases increase the degree and rate of crosslinking and as such present a challenge to maximizing health and lifespan. AGE crosslinks are formed when binding occurs between sugars (e.g., glucose) and lipids or proteins in the intracellular and extra cellular matrix. This affects nearly every cell in the body.

While crosslinking is a natural and necessary physiological process, excessive binding results in cell stiffening which results in them becoming more susceptible to damage and premature ageing. AGE crosslinking has been predominantly studied for its involvement in specific disease models (e.g., cardiovascular disease mortality and diabetes); however, it has also been proposed by the anti-aging community as one of the main causes of degenerative aging and senescence.

Accordingly, there is a need for compositions and methods to reduce AGE crosslinking.

### SUMMARY OF THE INVENTION

In one aspect, the invention provides a composition including two, three, or all of rosmarinic acid; alpha-lipoic acid; carnosine; and benfotiamine.

The amount of rosmarinic acid is for example 20-2000 mg, e.g., about 200 mg. The amount of alpha-lipoic acid is for example 30-3000 mg, e.g., about 300 mg. The amount of carnosine is for example 50-5000 mg, e.g., about 500 mg. The amount of benfotiamine is for example 25-2500 mg, e.g., about 250 mg.

The composition may further include vitamin B6, e.g., at 10-100 mg, such as about 30 mg.

The composition may further include one or more of vitamin C; blueberry and/or bilberry extract; resveratrol; curcumin; and taurine. The amount of vitamin C is for example 10-1000 mg, e.g., 100 mg. The amount of curcumin is for example 80-8000 mg, e.g., 800 mg. The amount of taurine is for example 40-4000 mg, e.g., about 400 mg. The amount of blueberry and/or bilberry extract is for example 30-3000 mg, e.g., about 300 mg. The amount of resveratrol is for example 25-2500 mg. e.g., about 250 mg.

The composition may also further include clove and/or cinnamon.

In a related aspect, the invention provides a kit including two, three, or all of rosmarinic acid; alpha-lipoic acid; carnosine; and benfotiamine, wherein two, three, or all of the rosmarinic acid; alpha-lipoic acid; carnosine; and benfotiamine are formulated in at least two compositions.

The amount of rosmarinic acid is for example 20 - 2000 mg, e.g., about 200 mg. The amount of alpha-lipoic acid is for example 30-3000 mg. e.g., about 300 mg. The amount of carnosine is for example 50-5000 mg, e.g., about 500 mg. The amount of benfotiamine is for example 25-2500 mg, e.g., about 250 mg.

The kit may further include vitamin B6, e.g., at 10-100 mg, such as about 30 mg.

The kit may further include one or more of vitamin C; blueberry and/or bilberry extract; resveratrol; curcumin; and taurine. The amount of vitamin C is for example 10-1000 mg, e.g., 100 mg. The amount of curcumin is for example 80-8000 mg, e.g.. 800 mg. The amount of taurine is for example 40-4000 mg, e.g., about 400 mg. The amount of blueberry and/or bilberry extract is for example 30-3000 mg, e.g., about 300 mg. The amount of resveratrol is for example 25-2500 mg, e.g., about 250 mg.

The kit may also further include clove and/or cinnamon.

In a further aspect, the invention provides a method of reducing AGE crosslinking or the visible signs of aging by administering an effective amount of a composition or kit of the invention to a subject in need thereof.

By “about” is meant +/−10% of the amount listed.

By “effective amount” is meant an amount sufficient to confer benefit, e.g., clinical benefit.

### DETAILED DESCRIPTION OF THE INVENTION

This invention combats many negative processes associated with aging. The science and understanding of aging has been expanding at a massive rate over the last decade. Breakthroughs in research in academic institutes and privately held pharmaceuticals are now describing and modelling the process on a molecular level. The identification of key proteins, enzymatic pathways and exo and endogenous biomolecules allows ever-increasing loci of opportunity to intercede with naturally occurring extracts that have been proven to have a discrete pharmacological effect. AGE crosslinking presents one of the challenges facing the population in terms of maximizing health span and dealing with internal issues that expedite the aging process. The present invention not only offers the most comprehensive support to both inhibit and break down AGE crosslinks but also deliver powerful results in antioxidant capacity and anti-inflammatory results.

The invention provides compositions, kits, and methods that are useful for treating the signs of aging.

### Compositions and Kits

A composition or kit for inhibiting AGE crosslinking, e.g., useful for treating the signs of aging, includes two, three, or all of the following components:

Rosmarinic acid, e.g., 20-2000 mg, e.g., 200 mg

Alpha-Lipoic acid, e.g., 30-3000 mg, e.g., 300 mg

Benfotiamine, e.g., 25 - 2500 mg, e.g.. 250 mg

Carnosine, e.g., 50-5000 mg, e.g., 500 mg

And optionally Vitamin B6, e.g., 10-100 mg. e.g., 30 mg.

Additionally, the composition or kit may include one or more of the following components:

Curcumin, e.g., 80-8000 mg, e.g., 800 mg

Taurine, e.g., 40-30000 mg, e.g.. 40-4000 mg, e.g.. 400 mg or 20 g

Blueberry and/or Bilberry extract, e.g., 30-3000 mg, e.g., 300 mg

Resveratrol. e.g., 25-2500 mg, e.g., 250 mg

Vitamin C, e.g.. 10-1000 mg. e.g., 100 mg

Alternatively, the composition or kit excludes one or all of these compositions.

In addition, the composition or kit may further include clove and/or cinnamon. The composition can be administered in a single dosage form containing all the listed ingredients or can be delivered in multiple dosage forms, each dosage form containing one or more of the listed ingredients. Thus, the invention provides both compositions and kits of multiple compositions including the ingredients. The composition or kit provides a synergistic effect in inhibiting AGE crosslinking.

Compositions may be formulated in any dosage form suitable for oral administration, e.g., tablets, pills, capsules, syrups, beverages, and combinations thereof, as is known in the art. A single composition may include or more of the ingredients, e.g., all of the ingredients or 1, 2, 3, 4, 5, 6, 7, 8, 9, 10, or 11 of the ingredients. When the ingredients are found in different compositions, they may be packaged together as a kit.

### Rosmarinic Acid

Rosmarinic acid is a caffeic acid ester of 3-(3,4-dihydroxyphenyl)lactic acid found in variety of plants. It has significant antioxidant properties. Rosmarinic acid has been shown to have a greater effect at reduction in AGE crosslinks than diabetic nephropathy drug candidate aminoguanidine or alagebrium. Rosmarinic acid has also shown promising results as a GABA transaminase inhibitor, which could have potential benefits for anxiety disorders and epilepsy. Furthermore, rosmarinic acid has shown antiviral and anti-inflammatory properties in a mouse model of Japanese encephalitis.

### Alpha Lipoic Acid (ALA)

ALA has been shown to protect against 4-hydroxynonenal (HNE) mediated oxidative stress and neurotoxicity, making it a potential adjunct therapy for Alzheimer's. HNE is a toxic and reactive α,β-unsaturated aldehyde formed by lipid peroxidation in cells with implication in neurodegenerative diseases, various cancers, diabetes and chronic inflammation. HNE, as a reactive carbonyl species can lead to the following AGEs: N-ϵ-carboxyethyl-lysine, arginine pyrimidine, pentosidine, pyrralin, methylglyoxal, glyoxal lysine and N-ϵ-carboxymethyl-lysine. ALA can mitigate AGE formations while simultaneously providing other benefits. ALA has also been shown to prevent collagen abnormalities in high fructose diets in rats.

### Benfotiamine

Benfotiamine is a synthetic derivative of Vitamin B1. It has been studied as a possible treatment for diabetic nephropathy, retinopathy, and neuropathy. Benfotiamine is an AGE formation inhibitor and has been shown to significantly attenuate the formation of AGEs and collagen crosslinking.

### L-Carnosine

L-Carnosine, an imidazole containing dipeptide formed by beta-alanine and histidine, is a naturally occurring compound found in red meat. This biologically active and important compound has beneficial antioxidative effects but more importantly helps prevent AGEs from forming by acting as an antiglycating agent. L-Carnosine has also been shown to reduce cellular senescence in a 96-patient trial in patients with cataracts, demonstrating an 80% success rate in advanced senile cataracts and a 100% success rate in patients with mild to moderate cataracts over the 6-month trial period. Carnosine is known as a geroprotector (i.e., a compound that can address the root causes of aging and consequently prolong life); it can also reduce the telomere shortening rate.

### Vitamin B6

Vitamin B6 has been heavily studied for its potential role in AGE and has shown efficacy in all common forms, such as pyridoxine, pyridoxal, and pyridoxamine. Additionally, pyridoxine has also shown improvements in diabetic patients with symptomatic peripheral neuropathy when used in conjunction with Vitamin B1. Vitamin B6 acts as a coenzyme function in roughly 100 enzyme reactions in glucose, amino acid, and lipid metabolism.

### Curcumin

Curcumin is a diarylheptanoid from the curcuminoid group of chemicals. It is a bright yellow compound found in turmeric and has a diverse pharmacology. However, its use is limited by poor oral bioavailability meaning that to be efficacious, more than two grams are required daily. A highly bioavailable, water soluble form is available, e.g., CurcuWIN. In addition to its antioxidant and anti-inflammatory properties, curcumin has also been shown to prevent the negative effects of AGE formations, specifically by countering AGE formation suppression of the Nrf2 pathway via RAGE induced gene expression of leptin and its receptors. Suppression of the Nrf2 pathway leads to increased oxidative stress which curcumin negated in a 2014 study.

### L-Taurine

L-Taurine has the ability to abolish fructose driven crosslinking in collagen; Fructose has been shown to be a more potent glycating agent than glucose in-vitro. Furthermore, it has been postulated that diabetic crosslink formations could be largely attributed to fructose in-vivo. Our current consumer climate is inundated with high fructose corn syrup as a sweetening agent, and L-taurine presents itself as an important supplement to reduce AGE crosslinks caused by fructose consumption.

### Bilberry or Blueberry Extract

Bilberry extract is included both for its own benefit in fighting AGEs, and also to complement L-Carnosine. The combination of carnosine and blueberry extract in vitro increased stem cell proliferation in vitro by 86%. Bilberries are a closely related Northern European berry that have a similar composition to blueberries and often show much higher levels of vitamins and anthocyanin. Anthocyanin is involved in glucose and lipid metabolism and has been shown to prevent the formations of AGEs by trapping methylglyoxal. It has also been suggested that bilberry can lower blood glucose levels and the naturally occurring flavonoids found in bilberries such as rutin and quercetin have been found to inhibit various stages of AGE formation. Oxerutin, a hydroxyethyl acetylation of rutoside(rutin), has boon shown to work in conjunction with taurine, which was found to be significantly more effective in a rodent model of diabetes resulting in reduced accumulation of collagen-linked fluorescence in skin.

### Resveratrol

Resveratrol is one of the most hyped and marketed putative anti-aging supplements to emerge in the last few decades and has been studied for its implications in life span, cancer, metabolism, cardiovascular issues and neurodegenerative issues. Its potential for reducing AGEs is less well reported however. A recent study found that Resveratrol inhibits AGEs induced proliferation and collagen synthesis activity. Resveratrol also shows positive results in studies pertaining to diabetes such as its effect in type II diabetic rats.

### Vitamin C

Ascorbic acid has also shown promise in fighting AGEs by decreasing serum protein glycation. It is a required cofactor for collagen synthesis and other critical enzymatic functions.

### Clove and Cinnamon

Clove and cinnamon contain high levels of eugenol and cinnamaldehyde in cinnamon. Eugenol, in addition to being anti-inflammatory has been shown to have a dual role in inhibition of AGEs. Cinnamaldehyde has likewise shown to attenuate AGEs by the suppression of AGE-induced biological responses mediated by inactivating the JAK2-STAT1/STAT3 cascade or activating the NO pathway. Cinnamaldehyde has also shown to exhibit anti-inflammatory and neuroprotective effects.

### Other Components

A composition of the invention may also include additional components, such as a, sweetener, flavoring, or coloring. Suitable sweeteners arc known in the art, e.g., sucrose, mannose, sucralose, aspartame, saccharin, stevia, and acesulfame K. A composition, e.g., tablet, may also include any food grade coloring and/or flavoring, such as a fruit flavoring.

### Methods of Use

The invention provides methods of reducing AGE crosslinking or the visible signs of aging by administering the ingredients described herein, e.g., formulated in one or more dosages. The administration may occur one or more times a day or with at least one day between administrations. Administration may be chronic, e.g., with repeated doses occurring over a period of at least on week, one month, three months, six months, or ono year. The methods of the invention may be employed on any appropriate subject. In one embodiment, the subject is at least 18 years old. e.g., at least 25, 35, 45, 55, 65, or 75 years old.

1. A composition comprising rosmarinic acid; alpha-lipoic acid; carnosine; and benfotiamine.

2. The composition of claim 1, wherein the amount of rosmarinic acid is 20-2000 mg.

3. (canceled)

4. The composition of claim 1, wherein the amount of alpha-lipoic acid is 30-3000 mg.

5. (canceled)

6. The composition of claim 1, wherein the amount of carnosine is 50-5000 mg.

7. (canceled)

8. The composition of claim 1, wherein the amount of benfotiamine is 25-2500 mg.

9. (canceled)

10. The composition of claim 1, further comprising one or more of vitamin C; blueberry and/or bilberry extract; resveratrol; curcumin; taurine; vitamin B6; clove; and/or cinnamon.

11. The composition of claim 10, wherein the amount of vitamin C is 10-1000 mg; wherein the amount of curcumin is 80-8000 mg; wherein the amount of taurine is 40-4000 mg; wherein the amount of blueberry and/or bilberry extract is 30-3000 mg; wherein the amount of resveratrol is 25-2500 mg.

12-21. (canceled)

22. A kit comprising: rosmarinic acid; alpha-lipoic acid; carnosine; and benfotiamine, wherein the rosmarinic acid; alpha-lipoic acid; carnosine; and benfotiamine are formulated in at least two compositions.

23. The kit of claim 22, wherein the amount of rosmarinic acid is 20-2000 mg.

24. (canceled)

25. The kit of claim 22, wherein the amount of alpha-lipoic acid is 30-3000 mg.

26. (canceled)

27. The kit of claim 22, wherein the amount of carnosine is 50-5000 mg.

28. (canceled)

29. The kit of claim 22, wherein the amount of benfotiamine is 25-2500 mg.

30. (canceled)

31. The kit of claim 22, further comprising one or more of vitamin C; blueberry and/or bilberry extract; resveratrol; curcumin; taurine; vitamin B6; clove; and/or cinnamon.

32. The kit of claim 31, wherein the amount of vitamin C is 10-1000 mg; wherein the amount of curcumin is 80-8000 mg; wherein the amount of taurine is 40-4000 mg; wherein the amount of blueberry and/or bilberry extract is 30-3000 mg; or wherein the amount of resveratrol is 25-2500 mg.

33-42. (canceled)

43. A method of reducing AGE crosslinking or the visible signs of aging, the method comprising administering an effective amount of a composition of claim 1 to a subject in need thereof.

44. The composition of claim 1, wherein the amount of rosmarinic acid is about 200 mg; the amount of alpha-lipoic acid is about 300 mg; the amount of carnosine is about 500 mg; or the amount of benfotiamine is about 250 mg.

45. The composition of claim 10, wherein the amount of vitamin C is about 100 mg; the amount of curcumin is about 800 mg; the amount of taurine is about 400 mg; the amount of blueberry and/or bilberry extract is about 300 mg; or the amount of resveratrol is about 250 mg.

46. The kit of claim 22, wherein the amount of rosmarinic acid is about 200 mg; the amount of alpha-lipoic acid is about 300 mg; the amount of carnosine is about 500 mg; or the amount of benfotiamine is about 250 mg.

47. The kit of claim 31, wherein the amount of vitamin C is about 100 mg; the amount of curcumin is about 800 mg; the amount of taurine is about 400 mg; the amount of blueberry and/or bilberry extract is about 300 mg; or the amount of resveratrol is about 250 mg.
